(19) United States
(12) Patent Application Publication
(10) Pub. No.: US 2022/0105000 A1
(43) Pub. Date: Apr. 7, 2022

(54) STABLE PARENTERAL DOSAGE FORM OF PYRAZOLONE DERIVATIVE

(71) Applicant: FTF PHARMA PRIVATE LIMITED, Ahmedabad (IN)

(72) Inventors: Jayanta Kumar MANDAL, Ahmedabad Gujarat (IN); Malay PATEL, Ahmedabad Gujarat (IN); Kartavya TRIVEDI, Kalol (IN); Rutul PATEL, Vadnagar (IN)

(73) Assignee: FTF PHARMA PRIVATE LIMITED, Ahmedabad (IN)

(21) Appl. No.: 17/493,397

(22) Filed: Oct. 4, 2021

(30) Foreign Application Priority Data
Oct. 5, 2020 (IN) 202021043170

(51) Int. Cl. A61J 1/06; A61J 1/14; A61K 31/4152; B65B 3/00; B65B 7/28; B65B 55/04

(52) U.S. Cl. CPC A61J 1/065; A61J 1/1412; B65B 55/04; B65B 3/003; B65B 7/28; A61K 31/4152

(57) ABSTRACT

Edaravone, in the United States, is approved in the form of ready-to-use intravenous solution for infusion, RADICAVA. RADICAVA injection is supplied in a polypropylene bag overwrapped with polyvinyl alcohol (PVA) secondary packaging. Edaravone is susceptible to oxidation and the overwrapped package also contains an oxygen absorber and oxygen indicator to minimize oxidation. Various efforts have been put in the art for packaging edaravone drug solution in different types of packaging materials, for example, plastic containers and glass bottles to prevent edaravone from oxidation and other degradation. The present invention involves use of simple and economic container-closure system for packaging edaravone drug solution while maintaining its stability during storage, transportation and use which includes glass bottles or vials and closures made of elastic polymer or rubber (for example elastomer, isoprene rubber or butyl rubber and the like).

### FIELD OF THE INVENTION

The present invention relates to a pharmaceutical dosage form preferably in the form of a parenteral dosage form which involves use of a packaging system which is stable during storage and transportation. The pharmaceutical dosage form of the present invention is convenient in use and involves use of less expensive packaging materials as compared to known packaging materials. The present invention also relates to a packaging system and packaging materials for packaging a parenteral dosage form of a drug which comprises use of bottles or vials, preferably made of glass and stoppers, preferably rubber stoppers which are uncoated rubber stoppers and optionally capping on said rubber stoppers.

### BACKGROUND OF THE INVENTION

The present invention is related to the field of pharmaceutical formulation technology. More particularly, the present invention is directed to the injectable pharmaceutical formulations of a drug suitable for parenteral administration. The drug which is used in the present invention is a pyrazolone derivative, viz. 3-methyl-1-phenyl-2-pyrazoline-5-one or its pharmaceutically acceptable salt.

The compound 3-methyl-1-phenyl-2-pyrazoline-5-one is a member of the substituted 2-pyrazoline-5-one class and is generically known as edaravone. The molecular formula of edaravone is C₁₀H₁₀N₂O and the molecular weight of edaravone is 174.20. The structural formula of edaravone is shown below.

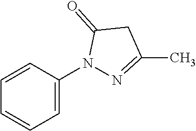

Edaravone is an active ingredient of the drug product RADICAVA, approved in the United States in May 2017 for the treatment of amyotrophic lateral sclerosis (ALS). Edaravone is a free radical scavenger that acts to scavenge various types of free radicals including active oxygen as a typical example, so as to prevent cell injury.

Amyotrophic lateral sclerosis (ALS), also known as motor neuron disease (MND) is a disease that causes the death of neurons controlling voluntary muscles. Some also use the term motor neuron disease for a group of conditions of which ALS is the most common. ALS is characterized by stiff muscles, muscle twitching, and gradually worsening weakness due to muscles decreasing in size. It may begin with weakness in the arms or legs, or with difficulty speaking or swallowing. About half of the people affected develop at least mild difficulties with thinking and behavior and most people experience pain. Most eventually lose the ability to walk, use their hands, speak, swallow, and breathe. U.S. Pat. No. 6,933,310 (assigned to Mitsubishi Tanabe Pharma Corporation) discloses and claims use of edaravone for the treatment of amyotrophic lateral sclerosis.

Edaravone was first approved in Japan as RADICUT. RADICUT has been commercially available as an injection 30 mg in the form of a 20 ml of solution containing 30 mg of edaravone filled into a glass ampoule.

RADICAVA injection is supplied for intravenous infusion in a polypropylene bag containing 30 mg/60 mg edaravone in 100 mL isotonic, sterile, aqueous solution, which is further overwrapped with polyvinyl alcohol (PVA) secondary packaging. The overwrapped package also contains an oxygen absorber and oxygen indicator to minimize oxidation, which should be pink to reflect appropriate oxygen levels. Each bag contains the following inactive ingredients: L-cysteine hydrochloride hydrate (20 mg), sodium bisulfate (40 mg). Sodium chloride is added for isotonicity and phosphoric acid and sodium hydroxide are added to adjust to pH 4.0.

The RADICAVA injection should be stored in overwrapped package to protect from oxygen degradation until time of use. The oxygen indicator will turn blue or purple if the oxygen has exceeded acceptable levels. Once the overwrap package is opened, RADICAVA injection should be used within 24 hours.

International Publication Number WO 2007/055312 (filed by Mitsubishi Tanabe Pharma Corporation) discloses a plastic container filled with an aqueous solution containing edaravone, coloration of which is suppressed. The '312 publication tested a bag using polyethylene, ethylene-vinyl acetate copolymer, polyvinyl chloride, polypropylene, or a combination of polyethylene and cyclic olefin polymer as the material, and ethylene. It is disclosed that the bag of vinyl acetate copolymer or polyvinyl chloride is colored. It is known that the use of cyclic polyolefin in the inner layer can prevent coloring of the formulation container by the active ingredient. However, a plastic container capable of suppressing a reduction in the content of edaravone due to adhesion of edaravone to the plastic container was not disclosed.

International Publication Number WO 2009/066752 (filed by Mitsubishi Tanabe Pharma Corporation) discloses a multilayered film and a plastic container each of which comprises a heat-sealable seal layer, a cyclic polyolefin layer and an outermost layer, wherein the seal layer comprises a polypropylene, the cyclic polyolefin layer comprises a cyclic polyolefin polymer or a cyclic polyolefin copolymer and the outermost layer comprises a layer comprising a polypropylene, and wherein each of the multilayered film and the plastic container further comprises a resin composition layer comprising a blend of a propylene polymer and a styrene-type elastomer. The plastic container enables to prevent the reduction in the content of a medicinal agent in a liquid pharmaceutical preparation, and has excellent impact resistance, excellent handling properties during filling a material into the container, excellent moldability, and excellent transparency.

The problem identified in the Japanese Patent Publication Number JP 2009084203 (Filed by Temuro Corp) is provision of a bottle, a bag or a prefilled syringe formulation made of a plastic stably housing an edaravone-containing aqueous formulation and to provide the bag formulation stably housing a premix formulation of the edaravone-containing aqueous formulation. To which, the '203 publication provides solution as a stable edaravone-containing aqueous formulation in which at least a part of a container housing the edaravone-containing aqueous formulation is a container made of a plastic composed of a cyclic polyolefin resin.

Japanese Patent Publication Number JP 2010162344 (filed by Fujimori Kogyo Co Ltd) discloses the package constituted by introducing the aqueous solution agent into a plastic container and sealing the container, thereafter packaging the container with a gas-barrier outer packaging material having a resin layer consisting essentially of a cyclic polyolefin-based resin, as an innermost layer or a layer adjacent to the innermost layer.

Chinese Patent Publication Number CN 101933899 (filed by Nanjing Simcere Dongyuan Pharmaceutical Co Ltd) discloses edaravone injection and a preparation method thereof. The injection contains edaravone serving as an active material, sodium pyrosulfite serving as antioxygen and propylene glycol serving as cosolvent. In the invention, in an edaravone injection production process, the sodium pyrosulfite antioxygen is added and high-purity nitrogen is charged into an ampoule bottle before and after liquid medicine canning and sealing to ensure the residual oxygen in the air above the liquid medicine in the ampoule bottle is less than 3 percent, so the edaravone is kept in a low-oxygen environment all the time. However, the ampoule packaging has several disadvantages. A small fixed volume of the drug does not allow to use the solution continuously (for example, with drip) in the desired volume. There is also a risk that small particles of glass getting into the infusion solution when the ampoule is opened.

When plastic is used, there is a problem of tightness, namely, oxygen from the air enters the solution of edaravone. Due to the high affinity edaravone combines with oxygen and changes the properties of the drug. To overcome this problem, antioxidants and double packaging are used to stabilize the edaravone solution, as described in the Japanese Patent Publication Number JP 2011136973 (Filed by Keiki Imoto et al.). That makes the process of obtaining a stable drug more complicate. The container is made of polypropylene, polyethylene or other flexible plastic. It has a rubber stopper of elastomer coated with a fluorine coating. The container is packed into another container having reduced oxygen permeability and made of a film based on aluminum oxide or silicon oxide or other similar material.

Japanese Patent Publication Number JP 2016022092 (filed by Nipro Corp) discloses a plastic container that is an alternative plastic container storing an aqueous solution containing edaravone, the plastic container showing sufficient stability. The plastic container disclosed in '092 publication is the plastic container having a polypropylene layer as an outer layer, a polypropylene layer as an intermediate layer, and a cyclic polyolefin layer as an inner layer.

International Publication WO 2019159967 (filed by Fujimori Kogyo Co Ltd) discloses a plastic container having superior storage stability of a pharmaceutical product, and in particular, a pharmaceutical formulation having a high affinity with plastics. The '967 publication more specifically discloses a plastic container in which an innermost layer of a sheet member forming a bag body of a bag is formed from an amorphous polymer, as a main component, by polymerizing at least one type or two or more types of olefin monomers, in which at least one type among the olefin monomers is a monomer having a cyclic hydrocarbon skeleton, and a port member forming an opening part is formed from a crystalline polyolefin having no cyclic hydrocarbon skeleton, as a main component.

International Publication Number WO 2020/044106 (filed by Sia Emteko Holding) discloses a method for producing a liquid dosage form of the drug Edaravone for parenteral use which is stable in storage and transportation and convenient for use, which involves: preparing a solution comprising Edaravone or pharmaceutically acceptable salts thereof as the active ingredient, and excipients (an acidic component, an alkaline component, an antioxidant, an osmolar agent and/or a stabilizer); packaging said dosage form in a pre-sterilized glass bottle having a cap at least partially coated with an anti-adhesive coating; sealing the bottle with said cap at least partially coated with an anti-adhesive coating, and sterilizing the bottle containing a solution comprising Edaravone or pharmaceutically acceptable salts thereof as the active ingredient, and excipients. The '106 publication further discloses a method for packaging a liquid dosage form of the drug Edaravone for parenteral use which is stable in storage and transportation and convenient for use, which involves: sterilizing a glass bottle having a cap; pouring a solution comprising Edaravone or pharmaceutically acceptable salts thereof as the active ingredient, and excipients (an acidic component, an alkaline component, an antioxidant, an osmolar agent and/or a stabilizer) into said sterilized glass bottle; closing (sealing) the bottle containing the solution using said cap, which is at least partially coated with an anti-adhesive coating; and sterilizing the sealed bottle containing the liquid dosage form. The '106 publication further discloses a bottle filled with a liquid dosage form of a drug for parenteral use comprising Edaravone or pharmaceutically acceptable salts thereof as the active ingredient, and excipients (an acidic component, an alkaline component, an antioxidant, an osmolar agent and/or a stabilizer), said bottle being made of glass and being closed with a cap which is made of a material based on flexible polymers and which is at least partially coated with an anti-adhesive coating.

It is evident from the prior art documents that storage of a drug solution containing pyrazolone derivative, i.e. edaravone and stability of edaravone in the solution during storage has always been prime concern of the researchers and for that various efforts have been put in. Discovery of plastic containers having multiple layers and different types of polymers and is one of them. Researchers have also found out that use of glass bottles or vials is also advantageous for the storage of edaravone solution during stability studies and shelf life. It is also convenient from the transportation view point. However, this research specifically talks about use of cap made of an elastic polymer and is coated, at least in part, with an anti-adhesive coating of polychlorotrifluoroethylene (PCTFE), perfluoroalkoxyalkane (PFA), ethylene tetrafluoroethylene (ETFE), fluoroethylene), polypropylene (FEP) or polyvinylidene fluoride (PVDF).

Therefore, the need still exists in the art, for more economic packaging system for storing edaravone drug solution which fulfills the purposes of stability of a drug in the solution during stability, shelf life and during transportation and ease and convenient in use.

The present inventors have, therefore, through their extensive research efforts, identified that when edaravone drug solution is filled in glass bottles or vials using simple and commonly available stoppers or caps which are not coated with anti-adhesive coating, still the above mentioned purposes can be achieved. It is well known in the industry that expensive packaging materials used for packaging the drug product ultimately increases the price of the drug and the whole burden is on the patient who has to pay such higher prices. Use of such uncoated stoppers or caps with glass bottles/vials make the present invention significantly economic without compromising with the quality of the drug product. With the packaging materials used in the present invention the price of the drug product may significantly be brought down.

### OBJECTS OF THE INVENTION

It is therefore one of the objects of the present invention to provide a drug solution containing a pyrazolone derivative, i.e. 3-methyl-1-phenyl-2-pyrazoline-5-one (edaravone) filled in the glass bottle or vials with the use of stoppers or caps without anti-adhesive coating.

A yet another object of the present invention is to use borosilicate glass bottles or vials and use of commonly available rubber stoppers (which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber and the like) or stoppers/caps made of elastic polymer. However, the stoppers/caps used in the present invention are uncoated which means they are without anti-adhesive coating.

A yet another object of the present invention is to pack the bottle or vial filled with edaravone drug solution with an aluminium cap over the stoppers (which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber and the like) which crimps the bottle cap in order to seal the container with the solution. The glass bottle or vial may further be packaged in simple and commonly available cartoon as secondary or outer packaging.

A yet another object of the present invention is to provide a container-closure system for packaging edaravone drug solution. A yet another object of the present invention is to provide a method for obtaining a parenteral dosage form of edaravone in the form of intravenous solution for infusion or drip, that is stable during storage, transportation and convenient in use. A yet another object of the present invention is to provide a packaging method for a parenteral dosage form of edaravone in the form of intravenous solution for infusion or drip, that is stable during storage, transportation and convenient in use.

A yet another object of the present invention is to prepare edaravone drug solution which comprises use of edaravone as an active ingredient, and a pharmaceutically acceptable excipient selected from an antioxidant, a stabilizer, a pH adjusting agent, a tonicity modifying agent, a vehicle and a combination thereof. The edaravone drug solution according to the present invention is in the form of ready-to-use parenteral dosage form for intravenous infusion.

### DETAILED DESCRIPTION OF THE INVENTION

The invention relates to the field of pharmaceutical formulation technology, in particular to a method for producing a drug product comprising a pyrazolone derivative 3-methyl-1-phenyl-2-pyrazoline-5-one, i.e. edaravone. The drug product of the present invention is stable during storage (stability and/or shelf life), transportation and use.

Edaravone has the ability to scavenge free radicals and this is why it is used as a scavenger of free radicals in cerebral infarction. Edaravone is administered by intravenous infusion or drip.

However, there is a significant problem when using edaravone, in particular, its instability in solution, adhesion of the molecules of the drug substance on the surface of the bottle cap. In addition, when using packaging with insufficient tightness, the concentration of edaravone in the solution increases due to the evaporation of the liquid.

In the prior art, the abovementioned problem has been tried to overcome by packing edaravone drug solution in glass ampoules to ensure hermetic storage of a solution with a constant concentration.

In order to get rid of residual oxygen in the space above the edaravone solution and to ensure the stability of the drug, nitrogen is added before sealing the ampoules with edaravone solution.

However, ampoule packaging has also numbers of disadvantages, including a small-fixed volume of the drug, which does not allow the solution to be used continuously in the required volume (for example, when the drug is used in infusion or drip). There is also a risk of small glass particles getting into the infusion solution when opening the ampoule. If proper precaution and care is not taken the opener or the health care professional opening the ampoule may also get injured.

An attempt has therefore been also made to solve above problem existing in the prior art by replacing the glass ampoule with a plastic container. However, such packaging usually has a complex structure. The plastic container is formed by an outer polypropylene layer, an intermediate polypropylene layer and an inner cyclic polyolefin layer.

A bottle, bag, or syringe pre-filled with the edaravone solution is also used. These packages are at least partially made of plastic, in particular of a cyclic polyolefin resin. A rubber stopper made of elastomer, isoprene rubber or butyl rubber is provided in the said plastic container, and the surface that contacts the edaravone solution is coated with a fluorine or parylene resin having anti-adhesive properties.

However, when using plastic, the problem of the tightness arises, namely, oxygen from the air enters into the edaravone solution. Due to the high affinity, when edaravone combines with atmospheric oxygen, the properties of the drug change. In order to overcome this problem, antioxidants and double packaging are used to stabilize the edaravone in the solution, which complicates the process of obtaining a stable finished dosage form. So, the container is made of polypropylene, polyethylene or other flexible plastic; it has a rubber stopper made of elastomer coated with a fluorine-based coating. The container is packaged in another container that has a reduced oxygen permeability and is made of a film based on alumina or silica or other similar material.

Although this package provides stability to the drug into the solution, it is not convenient for use, because after opening or damaging the seal of the outer container, it loses its protective property. In addition, such packaging does not provide containers of different sizes (volumes), which can lead to irrational use of the drug.

From the prior art documents, edaravone or its pharmaceutically acceptable salt in liquid form which is ready-to-use packaged in different packaging materials is known. Packaging in the form of ampoules is hermetically sealed, however, the disadvantage of this form of preparation is the impossibility of multiple use of one ampoule and the high probability of glass particles getting into the infusion solution. Also, the drug is produced in large volume plastic packaging, which, however, is not tight enough and, as a result, allows oxygen to pass from the environment, which negatively affects the quality of the drug.

A multilayer plastic container performs its function of ensuring the stability of the drug during storage and transportation only until it is opened. After opening the container, the drug must be used immediately, since the tightness is broken and the degradation of edaravone starts occurring. As a result, the drug increases the permissible level of impurities/degradants that can cause undesirable side effects and/or less therapeutic effect when used in patients. In addition, this form of packaging is complex both for the production of the drug itself (the plastic must withstand double high heat treatment with the drug), and for the production of packaging as such (a double container is made of a triple layer of plastic).

Thus, there is a problem of obtaining the edaravone in the solution packaged in such a package which would allow maintaining its stability during storage, transportation, and use.

Accordingly, the object of the present invention is to provide a ready-to-use, stable (during storage-stability/shelf life), easy and convenient to transport and use edaravone parenteral dosage form. The edaravone finished dosage form according to the present invention is in the form of intravenous solution for infusion or drip.

Accordingly, the present invention provides a method for obtaining a parenteral dosage form of edaravone in the form of intravenous solution for infusion or drip, that is stable during storage, transportation and convenient in use. The method comprises:
(a) preparing a solution containing edaravone or its pharmaceutically acceptable salt as an active ingredient and a pharmaceutically acceptable excipient selected from an antioxidant, a stabilizer, a pH adjusting agent, a tonicity modifying agent, a vehicle and a combination thereof;
(b) packaging the edaravone dosage form in a glass bottle or vial, preferably pre-sterilized, with a lid covered on said glass bottle or vial, wherein said lid is without an anti-adhesive coating; and
(c) closing the bottle or vial with a lid covered on said bottle or vial and sterilizing said bottle or vial containing edaravone solution.

In an alternative embodiment, the present invention provides a method for obtaining a parenteral dosage form of edaravone in the form of intravenous solution for infusion or drip. The method comprises:
(a) preparing a solution containing edaravone or its pharmaceutically acceptable salt as an active ingredient and a pharmaceutically acceptable excipient selected from an antioxidant, a stabilizer, a pH adjusting agent, a tonicity modifying agent, a vehicle and a combination thereof;
(b) packaging the edaravone dosage form in a glass bottle or vial, that is optionally pre-sterilized, with a lid covered on said glass bottle or vial, wherein said lid is without an anti-adhesive coating; and
(c) closing the bottle or vial with a lid covered on said bottle or vial and optionally sterilizing said bottle or vial containing edaravone solution.

In the above method, the glass bottle or vial is made of borosilicate glass and the lid or cap or stopper is made of an elastic polymer or rubber (which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber and the like) which is uncoated, i.e. without anti-adhesive coating.

The present invention also provides a packaging method for a parenteral dosage form of edaravone in the form of intravenous solution for infusion or drip, that is stable during storage, transportation and convenient in use. The method comprises:
(a) sterilization of a glass bottle or vial with a lid or cap over it;
(b) filling a solution containing edaravone or its pharmaceutically acceptable salt as an active ingredient and a pharmaceutically acceptable excipient selected from an antioxidant, a stabilizer, a pH adjusting agent, a tonicity modifying agent, a vehicle and a combination thereof into said pre-sterilized glass bottle or vial;
(c) closing (sealing) the bottle or vial containing edaravone solution with a lid or cap covered on it; and
(d) sterilizing the closed bottle or vial.

In an alternative embodiment, the present invention provides a packaging method for a parenteral dosage form of edaravone in the form of intravenous solution for infusion or drip. The method comprises:
(a) optionally sterilization of a glass bottle or vial with a lid or cap over it;
(b) filling a solution containing edaravone or its pharmaceutically acceptable salt as an active ingredient and a pharmaceutically acceptable excipient selected from an antioxidant, a stabilizer, a pH adjusting agent, a tonicity modifying agent, a vehicle and a combination thereof into said optionally pre-sterilized glass bottle or vial;
(c) closing (sealing) the bottle or vial containing edaravone solution with a lid or cap covered on it; and
(d) optionally sterilizing the closed bottle or vial.

In the above method, the glass bottle or vial is made of borosilicate glass and the lid or cap or stopper is made of an elastic polymer or rubber (which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber and the like) which is uncoated, i.e. without anti-adhesive coating.

The present invention further provides a bottle or vial filled with a parenteral dosage form of edaravone in the form of intravenous solution for infusion or drip, containing edaravone or its pharmaceutically acceptable salt as an active ingredient, and a pharmaceutically acceptable excipient selected from an antioxidant, a stabilizer, a pH adjusting agent, a tonicity modifying agent, a vehicle and a combination thereof, the bottle or vial made of glass, closed with a lid or cap or stopper made of a material based on an elastic polymer, rubber (which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber and the like) the lid or cap is not coated with an anti-adhesive coating.

According to the present invention, the bottle or vial is made of borosilicate glass, the lid or cap or stopper is made of a derivative of rubber or thermoplastic polymer and on the inside or is completely uncoated with a release coating known in the art. When packing, an aluminum cap may also be used, which crimps the bottle cap in order to seal the container with the solution containing edaravone.

### A Pharmaceutically Acceptable Excipient:

The edaravone formulation as described in the present invention comprises a pharmaceutically acceptable excipient, which comprises an antioxidant, a stabilizer, a pH adjusting agent, a tonicity modifying agent, a vehicle and a combination thereof. Non-limiting embodiments on the pharmaceutically acceptable excipients which may be used in the present invention are described below.

Antioxidants can be compounds that can reduce a drug that has been oxidized, or compounds that are more readily oxidized than the agents they are to protect (oxygen scavengers). Mixtures of chelating agents and antioxidants are often used because there appears to be a synergistic effect. This occurs because many of the agents act at differing steps in the oxidative process. The antioxidants when used in the compositions of the present invention include, but are not limited to, butylated hydroxytoluene, butylated hydroxyanisole, tert-butyl-hydroquinone, 4-hydroxymethyl-2, 6-di-tert-butylphenol, 2, 4, 5-trihydroxybutyrophenone, alkylgallates, propyl gallate, octyl gallate, dodecyl gallate, ethoxyquin, gallic acid, nordihydroguaiaretic acid, glycine, ascorbic acid, fatty acid esters of ascorbic acid such as ascorbyl palmitate and ascorbyl stearate, and salts of ascorbic acid such as sodium, calcium, or potassium ascorbate; erythorbic acid, L-carnitine, monothioglycerol, acetyl L-carnitine, thioglycolic acid, N-acetyl cysteine, cysteine, glutathione, methionine, tartaric acid, citric acid, fumaric acid, glycolic acid, oxalic acid, succinic acid, ellagic acid, malic acid, maleic acid, tocopherols such as, but not limited to, delta tocopherol, alpha tocopherol; lipoic acid, thiolated polymers such as, but not limited to, polymethacrylic-SH, carboxy methylcellulose-cysteine, polycarbophil-cysteine, beta-carotene, carotenoids, flavonoids, flavones, isoflavones, flavanones, catechins, anthocyanidins, chalcones, sulfites, including but not limited to potassium sulfite, sodium metabisulfite, sodium sulfite, sodium thiosulfate and sodium bisulfite or a combination thereof may be employed. The use of an antioxidant in the form of sodium bisulfite (about 0.005 mg/mL to about 1.0 mg/mL) has been found effectively prevents oxidation of edaravone in the solution at elevated temperature in pre-fillable syringes, ampoules and bottles/vials.

Typically, tonicity adjusting agents are used to adjust the osmolality of the pharmaceutical compositions to bring it closer to the osmotic pressure of body fluids, such as blood or plasma. In some embodiments the tonicity of the formulation can be modified by adjusting the concentration of buffer and/or other components present in the formulation.

Provided that the compositions are physiologically compatible, the compositions do not require any particular osmolality. Thus, the compositions can be hypotonic, isotonic or hypertonic. Typically, the pharmaceutical compositions have a tonicity between about 250 to about 350 mOsm/kg. In some of the alternative embodiments, the formulations of the present invention are isotonic, i.e., in the range of 260-320 mOsm/kg. In some of the alternative embodiments, the formulations of the present invention are isotonic, i.e., in the range of 270-328 mOsm/kg. However, the formulations may have a tonicity in the range of 250-350 mOsm/kg. Therefore, the formulations may be either slightly hypotonic, 250-269 mOsm/kg, or slightly hypertonic, 329-350 mOsm/kg.

Suitable tonicity adjusting agents for use in the pharmaceutical compositions include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts. The quantity of the tonicity adjusting agent in the formulation can be expressed in mg/ml or in g/L. In typical embodiments, the tonicity adjusting agent(s) is present from about 1 mg/ml to about 90 mg/ml. Thus, the pharmaceutical compositions can comprise one or more tonicity adjusting agents at about 1-5 mg/ml, at about 5-10 mg/ml, at about 10-15 mg/ml, at about 15-25 mg/ml, at about 25-50 mg/ml, at about 50-60 mg/ml, at about 60-70 mg/ml, at about 70-80 mg/ml, and at about 80 to 90 mg/ml, as well as combinations of the above ranges.

Alternatively, the tonicity adjusting agent concentration is measured in weight/volume percent. In typical embodiments, the tonicity adjusting agent(s) is present from about 0.1% to about 10%. For example, suitable tonicity adjusting agent concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8%, from about 0.8% to about 0.9%, from about 0.9% to about 1%, from about 1% to about 2%, from about 2% to about 3%, from about 3% to about 4%, from about 4% to about 5%, from about 5% to about 6%, from about 6% to about 7%, from about 7% to about 8%, from about 8% to about 9%, and from about 9% to about 10%, as well as combinations of the above ranges.

In some embodiments, the tonicity adjusting agent is sodium chloride. Typically, the concentration of sodium chloride suitable for use in the pharmaceutical compositions is between about 0.1% (w/v) to about 1.8%. By way of example, suitable sodium chloride concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8% (which is equivalent to 8 mg/ml), from out 0.8% to about 0.9% (which is equivalent to 9 mg/ml), from about 0.9% to about 1.0%, from about 1% to about 1.2%, from 1.2% (which is equivalent to 12 mg/ml) to about 1.4%, from about 1.4% to about 1.6%, and from about 1.6% to about 1.8%.

In some embodiments, the pharmaceutical composition comprises two, three, or more tonicity adjusting agents. In these embodiments, the concentration of each tonicity adjusting agent is typically less than the concentration that is used when only a single agent is present in the formulation.

Buffers/Buffering agent(s) if and when used in the formulation of the present invention include, but are not limited to, pharmaceutically acceptable salts and acids of acetate, glutamate, citrate, tartrate, benzoate, lactate, histidine or other amino acids such as arginine, alanine, glycine and lysine, gluconate, phosphate, malate, succinate, formate, propionate, and carbonate. “Pharmaceutically acceptable” is used herein in the sense of being compatible with the other ingredients of the formulation and not deleterious to the recipient thereof. Accordingly, the term “pharmaceutically acceptable salt” references salt forms of the active compounds which are prepared with counter ions which are non-toxic under the conditions of use and are compatible with a stable formulation. Buffers suitable for use in the formulations of the present invention also include, but are not limited to tris (hydroxymethyl)aminomethane (TRIS), triethanolamine, trolamine, diethanolamine, meglumine etc.

The concentration of the buffer/buffering agent in the formulation can be expressed in mg/ml, g/1 or as a molar concentration. Typically, from about 0.0001 mg/ml to about 100 mg/ml of a suitable buffer is present in the formulations of the present invention. Thus, the formulations can comprise from about 0.0001 to about 0.001 mg/ml of a suitable buffer, from about 0.001 to about 0.01 mg/ml of a suitable buffer, from about 0.01 to about 0.1 mg/ml of a suitable buffer, from about 0.1 to 1 mg/ml of a suitable buffer, from about 1 to about 5 mg/ml of a suitable buffer, from about 5 to about 10 mg/ml of a suitable buffer, from about 10 to about 15 mg/ml of a suitable buffer, from about 15 to about 20 mg/ml of a suitable buffer, from about 20 to about 25 mg/ml of a suitable buffer, from about 25 to about 50 mg/ml of a suitable buffer, from about 50 to about 75 mg/ml of a suitable buffer, and from about 75 to about 100 mg/ml of a suitable buffer.

Alternatively, the buffer concentration can be expressed as molar concentrations. In typical embodiments, from about 0.1 to 100 mM of a suitable buffer is present in the pharmaceutical compositions. Thus, the pharmaceutical compositions can comprise a suitable buffer having a concentration from about 0.1 to about 100 mM, from about 0.1 to about 0.5 mM, from about 0.5 to about 1.0 mM, from about 1.0 to about 5 mM, from about 5 to about 10 mM, from about 10 to about 15 mM, from about 15 to about 25 mM, from about 25 to about 50 mM, from about 50 to about 75 mM, and from about 75 to about 100 mM.

In some embodiments, the formulation of the present invention comprises a pH adjusting agent. Suitable pH adjusting agents typically include at least an acid or a salt thereof, and/or a base or a salt thereof. Acids and bases can be added on an as needed basis in order to achieve the desired pH. For example, if the pH is greater than the desired pH, an acid can be used to lower the pH to the desired pH. Acids suitable for use in formulations include, but are not limited to, hydrochloric acid, phosphoric acid, citric acid, ascorbic acid, acetic acid, sulphuric acid, carbonic acid, nitric acid and the like. By way of another example, if the pH is less than the desired pH, a base can be used to adjust the pH to the desired pH. Bases suitable for use in formulations include, but are not limited to, sodium hydroxide, potassium hydroxide, calcium hydroxide, sodium carbonate, sodium citrate, sodium acetate, magnesium hydroxide and the like.

A “Buffer/Buffering agent” or a “pH adjusting agent” as used herein is a system which is used for the purposes and is capable of maintaining the desired/required pH of the formulations throughout desired/required time period, e.g. stability studies and/or shelf life of the drug product. The desired pH of the formulation according to the present invention is about 4.0.

In some of the embodiments of the invention, both buffering agent and pH adjusting agent is used. In some of the embodiments of the invention, only a buffering agent is used. In some of the embodiments of the invention, only a pH adjusting agent is used.

In some of the preferred embodiments, phosphoric acid and/or sodium hydroxide are used as pH adjusting agent(s) which adjusts and/or maintains the desired/required pH, i.e. about 4.0.

However, in the non-limiting embodiments of the present invention, the pH of the edaravone formulation is between about 3.0 and about 5.0. In some of the non-limiting embodiments, the pH of the edaravone solution of the present invention is about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, and about 5.0. In some of the further non-limiting embodiments, the pH of the edaravone solution of the present invention is any value between the ranges about 3.0 to about 5.0.

Stabilizing agents when used in the formulation of the present invention without limitation include ethylene diamine tetraacetic acid (EDTA) or salts thereof, ascorbic acid, cysteine or salt thereof, L-cysteine or salt thereof, sorbitol, sorbitol solution, glycols such as propylene glycol and the like and a combination thereof. The use of a stabilizing agent in the form of L-cysteine hydrochloride hydrate (about 0.005 mg/mL to about 1.0 mg/mL) has been found effectively stabilizes edaravone in the solution at elevated temperature in pre-fillable syringes, ampoules and bottles/vials.

Since, the edarvone formulation of the present invention is in the form of aqueous solution, water or water for injection is used as a vehicle.

Further, during the packaging of the drug product of the present invention and also during its preparation, the drug product may have to undergo sterilization process for multiple times, for example, twice. Therefore, the materials from which the container which contains ready-to-use edaravone injection solution is made, must have appropriate characteristics.

If above mentioned conditions are fulfilled then prolonged stability of the drug (i.e. edaravone) may be ensured during its storage under various conditions and may make the edaravone solution prepared according to the present invention safer for use, storage for prolonged time and even during transport.

The packaging material according to the present invention, which should ensure the stability of the drug into the solution without changing pH, density, heat resistance and other characteristics, is borosilicate glass, which can easily withstand temperatures above 200° C. and does not change the chemical composition of the edaravone. Thus, it is advantageous to fill or store the edaravone solution in a borosilicate glass container.

The closure of the glass bottle or vial with the edaravone drug product must be carried out in such a way that the lid or cap or stopper, firstly, seals the vial hermetically, and secondly, the lid or cap or stopper must be heat-resistant, and thirdly, made of a material that prevents the adhesion of the active substance on its surface.

According to the present invention, the lid or cap or stopper is made of an elastic polymer based on a rubber or thermoplastic derivative, which has high impermeable properties, high heat resistance, strength and at the same time good flexible properties, which prevents the lid from breaking. Other similar materials may also be used which meet the specified characteristics, for example, in the class of unnatural synthetic elastomers. It is pertinent to note that the lid or cap or stopper used in the present invention are uncoated and does not contain anti-adhesive coating.

It is also not required that at least that part of the surface of the lid or cap or stopper, partially or completely, which is in contact with the drug solution, be covered with a layer of a substance with anti-adhesive properties. The lid or cap or stopper should not affect the chemical composition of the mixture and it should be heat-resistant.

During transportation and storage, additional stability of the drug in the package of the present invention is due to the use of an aluminum cap, which crimps the bottle or vial cap to seal the container containing edaravone solution.

As primary packaging materials, the present invention uses glass vials, rubber closures and aluminum tear off seals. The glass vials used in the present invention are USP/EP Type-I clear 100 mL ISO-Molded glass vials (manufactured by SGD Pharma India Ltd.). However, other glass vials of similar quality and having different size (capacity) may also be used for the purposes of the present invention. Similarly, Type II and Type III glass containers may also be used if found suitable for parenteral use. The glass vials of the present invention are made of borosilicate glass. However, vials made of other glass material (which, for example, without limitation include lime soda glass, neutral glass, lead free glass sulphured containers, silicone treated containers and the like) may also be used if found suitable for parenteral use. The lids or caps or stoppers used in the present invention to close the vials containing edaravone solution are 20 mm chlorobutyl rubber stoppers (S-127 4432/50/Grey Westar RS SIL 1; manufactured by West Pharmaceutical Services Singapore Pte. Ltd.). However, 20 mm-35 mm size rubber closures which, for example, without limitation include natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, chloroprene rubber or any other suitable closures made of suitable material and the like may also be used for the purposes of the present invention. The formulation of the rubber closures used in the present invention complies with the chemical requirements in the corresponding monograph 3.2.9 for “rubber closures” Type 1 in the valid version of the European Pharmacopoeia. The formulation also complies with the physicochemical tests, as described in the current USP General Chapter [381] “Elastomeric Closures for Injections”. The rubber closures used in the present invention are uncoated which means they do not contain any anti-adhesive coatings. The present invention also uses 20 mm size Aluminum tear off seal to pack the vial containing edaravone solution. These Aluminum seal crimps the vial cap to seal the containers. However, Aluminum seals of any suitable size may also be used depending upon the size of the stopper used.

As secondary packaging materials, the present invention uses white folded cartoon. However, any packaging material made of suitable material which fulfills the requirements of the present invention (i.e. stability during storage and transportation, easy and convenient use etc.) may also be used.

### Definitions

The use of the terms “a” and “an” and “the” and similar referents in the context of describing the invention are to be construed to cover both the singular and the plural, unless otherwise indicated herein or clearly contradicted by context.

As used herein, the term “about” is synonymous with “approximately” and is used to provide flexibility to a numerical value or range endpoint by providing that a given value may be “a little above” or “a little below” the value stated. “About” can mean, for example, within 3 or more than 3 standard deviations. “About” can mean within a percentage range of a given value. For example, the range can be ±1%, ±5%, ±10%, ±20%, ±30%, ±40% or ±50% of a given value. “About” can mean with an order of magnitude of a given value, for example, within 2-fold, 3-fold, 4-fold or 5-fold of a value. However, it is to be understood that even when a numerical value is accompanied by the term “about” in this specification, that express support shall be provided at least for the exact numerical value as well as though the term “about” were not present.

As used herein, the terms “stable” or “stability” encompass any characteristic of the formulations which may be affected by storage conditions including, without limitation, potency, total impurities, degradation products, specific optical rotation, optical purity, water content, appearance, viscosity, sterility, and colour and clarity. The storage conditions which may affect stability include, for example, duration of storage, temperature, humidity, and/or light exposure.

The term “degradant”, “impurity”, “degradation impurity” and “related substance” as used herein represents the same meaning and can be used interchangeably.

In some embodiments, the formulations of the present invention are stable for prolonged time when stored under storage conditions. The term “storage conditions” as used herein without limitation include typical storage conditions such as 2° C.-8° C. The compositions of the present invention may also be stored at 40° C.±2° C./75±5% RH, 30° C.±2° C./65±5% RH, 25° C.±2° C./40±5% RH, 25° C.±2° C./60±5% RH, 40° C.±2° C./NMT 25% RH (NMT=not more than) and accelerated conditions such as 40° C.±2° C./75±5% RH. The term “prolonged time” as used herein indicates that the formulations of the present invention are stable for at least 1 month or more, at least 3 months or more, at least 6 months or more or at least 12 months or more when stored under storage conditions.

In some of the embodiments of the present invention, “stable” or “storage stable”, or “stability” when used with reference to the formulations of the present invention or when used “stable or storage stable formulations” or “stability of the formulations” all these terms/phrases refer to formulations of the present invention which retain at least about 90%, or at least about 95%, or at least about 96%, or at least about 98%, of the labelled concentration of edaravone or salt thereof contained in the said formulation after storage under typical and/or accelerated conditions. In further embodiments, stable formulations or stability of the formulations refer to less than about 15% (area percent), or less than about 10% (area percent), or less than about 7% (area percent), or less than about 5% (area percent), or less than about 2% (area percent) of edaravone-related impurities are present after storage under typical and/or accelerated conditions.

In some of the embodiments, formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) any known or unknown single edaravone-related impurity or other impurity after storage under typical and/or accelerated conditions.

In some of the embodiments, formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) total edaravone-related impurities or other impurities after storage under typical and/or accelerated conditions.

Methods for determining the stability of the formulations of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the formulations are determined by a peak area percent method using HPLC.

The term “compatible” as used herein refers to those added excipients or ingredients or additives those are not substantially antagonistic to the other excipients or ingredients or additives or pharmaceutically active ingredients.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures.

As used herein, “optional” or “optionally” means that the subsequently described event or circumstance does or does not occur, and that the description includes instances where said event or circumstance occurs and instances where it does not. For example, an optionally substituted group means that the group is un-substituted or is substituted.

As used herein, “comprises”, “comprising”, “containing” and “having” and the like can have the meaning ascribed to them in patent law and can mean “includes”, “including” and the like, and are generally interpreted to be open ended terms. The terms “consisting of” or “consists of” are closed terms, and include only the components, structures, steps, or the like specifically listed in conjunction with such terms, as well as that which is in accordance with patent law. “Consisting essentially of” or “consists essentially of” have the meaning generally ascribed to them by patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including” it is understood that direct support should be afforded also to “consisting essentially of” language as well as “consisting of” language as if stated explicitly and vice versa. In essence, use of one of these terms in the specification provides support for all of the others. The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

As used herein, “rubber” without limitation includes natural rubber (latex), synthetic rubber, isoprene rubber (a chemical copy of natural rubber), styrene-butadiene rubber, neoprene (poly-(2-chloro-1,3-butadiene), styrol-butadine rubber, ethylene propylene dyes monomers, silicone (polysiloxane) rubber, butyl or halogenized butyl rubber such as bromobutyl, chlorobutyl and the like, nitrile rubber, and chloroprene rubber. Thus, the rubber stoppers include all stoppers made of above mentioned rubbers.

As used herein, “glass vials” or “glass bottles” without limitation include vials or bottles made of borosilicate glass, lime-soda glass, neutral glass, lead free glass, sulphured treated glass, and silicone treated glass. The glass vials according to the present invention without limitation include tubing glass vials and molded glass vials.

The term “easy and convenient to transport and use”, as used herein, means that the bottles or vials containing edaravone formulation of the present invention when packaged as described in the present invention are not adversely affected or damaged during transportation from one place to another and even during use, e.g., use by pharmacies, healthcare providers etc. In other words, the transportation and use of the edaravone formulation of the present invention when packaged as described in the present invention does not require any special handling techniques and/or care.

Further, as used herein, the term “stable during storage, transportation and convenient in use” means that the bottles or vials containing edaravone formulation of the present invention when packaged as described in the present invention, the quality of the product is not adversely affected or diminished during storage period or during transportation during one place to another and also during shelf life and that it is convenient to use by the healthcare providers or in pharmacies that no special handling techniques or care is required.

As used herein, “USP” is an abbreviated form of United States Pharmacopeia and “EP” is an abbreviated form of European Pharmacopeia.

All percentages mentioned herein, unless otherwise indicated, are on a w/v basis, i.e. percentage ingredient (active/inactive) present by weight in the total volume of the composition.

### BEST MODE OF CARRYING OUT THE INVENTION

### Example(s)

The edaravone parenteral dosage form of the present invention is prepared as ready-to-use intravenous solution for infusion. Said edaravone dosage form contains edaravone or its pharmaceutically acceptable salt as active ingredient and an antioxidant, a stabilizer, a pH adjusting agent and a tonicity modifying agent as inactive ingredients. The edaravone dosage form of the present invention is in the form of aqueous solution having pH about 4.0.

TABLE 1
Edaravone aqueous solution of the present invention

| | Name of | Role of | Strength(s) | |
|---|---|---|---|---|
| Sr | ingredient | ingredient | 0.3 mg/mL | 0.6 mg/mL |
| 1 | Edaravone | Active | 30 mg/100 mL | 60 mg/100 mL |
| 2 | L-cysteine | Stabilizer | 10 mg/100 mL | 20 mg/100 mL |
| | hydrochloride | | | |
| | hydrate | | | |
| 3 | Sodium bisulfite | Antioxidant | 20 mg/100 mL | 40 mg/100 mL |
| 4 | Sodium chloride | Tonicity | 860 mg/100 mL | 855 mg/100 mL |
| | | modifying | | |
| | | agent | | |
| 5 | Phosphoric | pH | Q.S. to | Q.S. to |
| | acid/sodium | adjusting | adjust/maintain | adjust/maintain |
| | hydroxide | agent(s) | pH about 4.0 | pH about 4.0 |
| 6 | Water for | Vehicle | Q.S. to 100 | Q.S. to 100 mL |
| | injection | | mL | |

Q.S. = quantity sufficient

The edaravone solution of the present invention, in the qualitative and quantitative composition as mentioned above, was prepared by dissolving required quantity of L-cysteine hydrochloride in the required quantity of the water for injection (having dissolved oxygen below 1 ppm). Required quantity of edaravone was dissolved in above solution. The mixture was heated, if required. Required quantity of sodium chloride was dissolved in the above drug solution. The pH was adjusted to about 4.0 using suitable pH adjusting agent (phosphoric acid/sodium hydroxide). For example, if pH of the solution is higher than 4.0 then pH is adjusted using phosphoric acid and if pH of the solution is lower than 4.0 then pH is adjusted using sodium hydroxide. Finally, required quantity of sodium bisulfite is dissolved in above pH adjusted drug solution. The pH of the final drug solution is checked and adjusted to about 4.0, if required, as mentioned above. The above method of preparation is for illustrative purpose only and the edaravone solution of the present invention can be prepared by following alternative methods also where the order of addition of drug and excipients is altered. The edaravone solution can also be prepared in two or more parts preparation process where some excipients with or without drug are dissolved in one part and some excipients with or without drug is dissolved in another part followed by mixing of both the parts to finally prepare edaravone solution. It should be noted that the order to addition of drug and excipients and the two-part preparation does not negatively affect the quality of the product.

The fact that sodium chloride used in the edaravone formulations of the present invention is 8.6 mg/mL (for 0.3 mg/mL strength) and 8.55 mg/mL (for 0.6 mg/mL strength) does not necessarily mean it can only be used in the edaravone formulations in these amounts only. Sodium chloride can be used in any quantity that is sufficient to bring the osmolality of the solution between about 260 mOsmol/kg and about 320 mOsmol/kg, e.g., sodium chloride may be used from 710 mg/100 mL to 860 mg/100 mL for both the strengths.

The edaravone drug formulation obtained by above method is filled in USP/EP Type-I clear 100 mL ISO-Molded glass vial (manufactured by SGD Pharma India Ltd.). The glass vial was closed using 20 mm chlorobutyl uncoated rubber stopper (S-127 4432/50/Grey Westar RS SIL 1; manufactured by West Pharmaceutical Services Singapore Pte. Ltd.). The glass vial closed with above said rubber stopper was finally clogged with 20 mm Aluminum tear off seal to pack the vial. The Aluminum seal crimps the vial cap to seal the container. The sealed vial was finally packed in white folded cartoon.

The stability of the edaravone in the solution using above mentioned primary and secondary packaging was tested at various temperatures and under different humidity conditions at various end points (for example after 1 month, 2 months, 3 months, 6 months, 9 months etc.). The results of these stability studies show that the edaravone was not susceptible to oxidation and other degradation even after using above mentioned simple and economic packaging materials. The concentration of edaravone in the solution was not increased due to evaporation of the liquid of the solution. Also, the concentration of edaravone was not decreased due to adhesion of the drug molecule with the rubber closure used even after using uncoated rubber stopper (rubber stopper without anti-adhesive coating). These surprising findings are not envisaged from the disclosure of any of the prior art documents, then, when the prior art documents specifically teaches advantages of the coated stoppers/lids with anti-adhesive polymers.

Thermal Stability Study Results of Edaravone Injection 30 mg/100 mL:

| | | 40° C./75% RH | | | 30° C./65% RH | | | | 25° C./60% RH | | | |
|---|---|---|---|---|---|---|---|---|---|---|---|---|
| | | 1 M | 3 M | 6 M | 1 M | 3 M | 6 M | 12 M | 1 M | 3 M | 6 M | 12 M |
| Test parameters | Initial | I | I | I | I | I | I | I | I | I | I | I |
| Description | C.S. | C.S. | C.S. | C.S. | C.S. | C.S. | C.S. | C.S. | C.S. | C.S. | C.S. | C.S. |
| pH | 4.25 | 4.21 | 3.93 | 3.68 | 4.30 | 4.09 | 3.64 | 3.63 | 4.02 | 4.02 | 3.67 | 3.65 |
| Osmolality | 280 | 283 | 287 | 282 | 280 | 288 | 280 | 282 | 284 | 288 | 281 | 283 |
| Assay of Edaravone | 100.1 | 99.7 | 98.7 | 98.0 | 99.8 | 99.3 | 98.8 | 99.3 | 99.9 | 100.1 | 99.4 | 100.2 |
| (in %) | | | | | | | | | | | | |
| Content of L- | 103.6 | 97.6 | 82.9 | 83.6 | 97.8 | 84.9 | 78.9 | 70.2 | 101.0 | 90.4 | 80.1 | 67.2 |
| Cysteine HCl | | | | | | | | | | | | |
| Mono-hydrate | | | | | | | | | | | | |
| (in %) | | | | | | | | | | | | |
| Content of Sodium | 89.1 | 82.4 | 73.6 | 66.9 | 84.8 | 77.4 | 70.0 | 64.8 | 86.9 | 81.2 | 70.3 | 65.7 |
| Bisulfite (in %) | | | | | | | | | | | | |
| Head Space Oxygen | 2.73 | 2.09 | 1.49 | 1.11 | 2.43 | 1.53 | 0.95 | 1.66 | 1.88 | 1.45 | 2.01 | 1.11 |
| (in % v/v) | | | | | | | | | | | | |
| Single maximum | 0.02 | 0.03 | 0.07 | 0.13 | 0.02 | 0.02 | 0.05 | 0.09 | 0.02 | 0.02 | 0.03 | 0.05 |
| unspecified | | | | | | | | | | | | |
| impurity | | | | | | | | | | | | |
| Total impurities | 0.06 | 0.12 | 0.23 | 0.44 | 0.07 | 0.07 | 0.17 | 0.38 | 0.06 | 0.06 | 0.12 | 0.20 |

C.S. = colorless solution;
NMT = not more than;
NLT = not less than;
RH = relative humidity;
M = month(s);
I = inverted

Thermal Stability Study Results of Edaravone Injection 60 mg/100 mL:

| | | 40° C./75% RH | | | 30° C./65% RH | | | 25° C./60% RH | | |
|---|---|---|---|---|---|---|---|---|---|---|
| | | 1 M | 3 M | 6 M | 1 M | 3 M | 6 M | 1 M | 3 M | 6 M |
| Test parameters | Initial | I | I | I | I | I | I | I | I | I |
| Description | C.S. | C.S. | C.S. | C.S. | C.S. | C.S. | C.S. | C.S. | C.S. | C.S. |
| pH | 4.24 | 3.59 | 3.78 | 3.54 | 3.85 | 3.59 | 3.57 | 3.95 | 3.67 | 3.60 |
| Osmolality | 286 | 287 | 293 | 289 | 287 | 293 | 288 | 288 | 292 | 288 |
| Assay of Edaravone | 99.8 | 99.2 | 99.1 | 97.9 | 99.5 | 99.6 | 98.9 | 99.6 | 99.8 | 99.0 |
| (in %) | | | | | | | | | | |
| Content of L-Cysteine | 102.3 | 96.3 | 88.0 | 81.5 | 98.3 | 77.9 | 82.7 | 101.1 | 83.3 | 83.5 |
| HCl Monohydrate (in %) | | | | | | | | | | |
| Content of Sodium | 80.9 | 76.6 | 68.9 | 64.9 | 78.7 | 64.5 | 67.2 | 80.5 | 66.8 | 68.4 |
| Bisulfite (in %) | | | | | | | | | | |
| Head space Oxygen | 3.59 | 1.54 | 0.89 | 3.66 | 2.07 | 1.34 | NP | 2.01 | 1.7 | NP |
| (in % v/v) | | | | | | | | | | |
| Single maximum | 0.02 | 0.04 | 0.07 | 0.14 | 0.03 | 0.04 | 0.07 | 0.03 | 0.05 | 0.03 |
| unspecified impurity | | | | | | | | | | |
| Total impurities | 0.04 | 0.11 | 0.18 | 0.38 | 0.07 | 0.11 | 0.17 | 0.07 | 0.11 | 0.09 |

C.S. = colorless solution;
NMT = not more than;
NLT = not less than;
RH = relative humidity;
M = month(s);
NP = not performed;
I = inverted

From above data it can be concluded that the edaravone formulation of the present invention when packaged in the packaging materials described herein is stable at 25° C. temperature and 60% relative humidity. Further, based on the above data it can be concluded that the shelf life of the edaravone solution when packaged in the packaging materials of the present invention can be at least 2 years (or 24 months) at 25° C.

It is pertinent to note that, the quality and stability of the drug product is not affected even after using uncoated stoppers/caps (that does not contain anti-adhesive coatings/polymers as described in the prior art). In other words, all the test parameters were within the specification even after using the uncoated stoppers/vials and keeping the containers containing edaravone solution in inverted position for above said time periods and stability conditions. These studies suggest that for packaging edaravone solution expensive packaging system (coated stoppers/caps having anti-adhesive polymers) is not required and the edaravone solution can be packaged in less expensive and easily available packaging materials which makes the drug product more economically viable and affordable by the patients.

The compositions and the packaging materials in which they are packaged as described in the present invention are suitable for use in the industry.

It should be understood that various changes and modifications to the embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention.

What is claimed is:

1. A bottle or vial filled with a parenteral dosage form of edaravone in the form of an intravenous solution for infusion or drip comprising edaravone or its pharmaceutically acceptable salt and a pharmaceutically acceptable excipient selected from an antioxidant, a stabilizer, a pH adjusting agent, a tonicity modifying agent, a vehicle and a combination thereof, the bottle or vial, closed with a lid or cap or stopper, wherein the lid or cap or stopper is not coated with an anti-adhesive coating.

2. The bottle or vial filled with a parenteral dosage form of edaravone as claimed in claim 1, wherein the bottle or vial is made of borosilicate glass.

3. The bottle or vial filled with a parenteral dosage form of edaravone as claimed in claim 1, wherein the lid or cap or stopper is made of a material based on an elastic polymer or rubber.

4. The bottle or vial filled with a parenteral dosage form of edaravone as claimed in claim 3, wherein the rubber is natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, or chloroprene rubber and the like.

5. The bottle or vial filled with a parenteral dosage form of edaravone as claimed in claim 1, wherein said parenteral dosage form is stable at 25° C. at least for 6 months or more or 12 months or more.

6. A method for obtaining a parenteral dosage form of edaravone in the form of an intravenous solution for infusion or drip, said method comprises:
(a) preparing a solution comprising edaravone or its pharmaceutically acceptable salt and a pharmaceutically acceptable excipient selected from an antioxidant, a stabilizer, a pH adjusting agent, a tonicity modifying agent, a vehicle and a combination thereof;
(b) packaging the edaravone dosage form in a glass bottle or vial, that is optionally pre-sterilized, with a lid or cap or stopper covered on said glass bottle or vial, wherein said lid or cap or stopper is without an anti-adhesive coating; and
(c) closing the bottle or vial with a lid or cap or stopper covered on said bottle or vial and optionally sterilizing said bottle or vial containing said edaravone solution.

7. The method for obtaining a parenteral dosage form of edaravone as claimed in claim 6, wherein the glass bottle or vial is made of borosilicate glass.

8. The method for obtaining a parenteral dosage form of edaravone as claimed in claim 6, wherein the lid or cap or stopper is made of rubber selected from natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, or chloroprene rubber and the like.

9. A packaging method for a parenteral dosage form of edaravone in the form of an intravenous solution for infusion or drip, said method comprises:
(a) optionally sterilization of a glass bottle or vial with a lid or cap or stopper over it;
(b) filling a solution comprising edaravone or its pharmaceutically acceptable salt and a pharmaceutically acceptable excipient selected from an antioxidant, a stabilizer, a pH adjusting agent, a tonicity modifying agent, a vehicle and a combination thereof into said optionally pre-sterilized glass bottle or vial;
(c) closing (sealing) the bottle or vial containing edaravone solution with a lid or cap or stopper covered on it; and
(d) optionally sterilizing the closed bottle or vial,
wherein said lid or cap or stopper is not coated with an anti-adhesive coating.

10. The packaging method for a parenteral dosage form of edaravone as claimed in claim 9, wherein the glass bottle or vial is made of borosilicate glass and the lid or cap or stopper is made of rubber selected from natural rubber (latex), synthetic rubber, isoprene rubber (a chemical copy of natural rubber), styrol-butadine rubber, ethylene propylene dyes monomers, silicone (polysiloxane) rubber, butyl or halogenized butyl rubber such as bromobutyl, chlorobutyl and the like, nitrile rubber, or chloroprene rubber and the like.
